(19) United States
(12) Patent Application Publication
(10) Pub. No.: US 2010/0089397 A1
(43) Pub. Date: Apr. 15, 2010

(54) BREATH RESPONSIVE POWERED AIR PURIFYING RESPIRATOR APPARATUS

(76) Inventor: Martinus Oliver Klockseth, Bromma (SE)

Correspondence Address: DICKSTEIN SHAPIRO LLP, 1825 EYE STREET NW, Washington, DC, 20006-5403, US

(21) Appl. No.: 12/517,523

(22) PCT Filed: Dec. 7, 2007
(86) PCT No.: PCT/SE07/01093

§ 371 Date: Jun. 3, 2009

(30) Foreign Application Priority Data
Dec. 12, 2006 (SE) 0602657-9

(51) Int. Cl. A61M 16/00; A62B 7/10

(52) U.S. Cl. 128/204.23; 128/205.12

(57) ABSTRACT

Breathing responsive powered air purifying respirator apparatus comprising an air-tight housing having connections for filters (5) and for supply of purified air to a face mask (3), a motor-driven blower arranged inside said air-tight housing for drawing air through said filter units and for the supply of purified air to the face mask. The blower is a lobe blower (6) delivering a constant flow of air. A regulator (10) is arranged in said housing in parallel with the lobe blower (6), and a by-pass conduit (11) connects the outlet side (12) of the lobe blower to the inlet side (13) of the lobe blower, wherein said regulator is arranged to open said by-pass conduit, when a pressure in the face mask exceeds a predetermined positive pressure.

The invention relates to a respirator apparatus, and more particularly to a breath responsive powered air purifying respirator.

### BACKGROUND

In the publication WO 2004/093997 is disclosed a breathing apparatus for assisting persons to breath in hostile environments, combining a so called Self Contained Breathing Apparatus (SCBA) and a Powered Air Purifying Respirator (PAPR).

In the publication WO 2005/055912 is disclosed a PAPR in which an electronic, pneumatic or optoelectronic performance control system controls the airflow from the blower to the face mask. Signals generated in the system are interpreted in a processor. The processor transmits signals to turn-on/turn-off the blower to increase/decrease the airflow from the blower.

In existing PAPRs, the blower has a rotating impeller, and the airflow is regulated by turning off/on the electric motor, which drives the impeller.

There are also other drawbacks with existing PAPRs. One drawback is that the efficiency of such blowers is quite low and because there is a significant pressure drop in the air flow line and valves, the blower has to work with a pressure well above the pressure required in the face mask. Accordingly, a battery will be drained quite quickly, despite the possibility of regulating the rotation speed of the blower.

Another problem is the capacity of the blower. For normal breathing, an airflow of about 60 l/min or 120 l/min would be sufficient to maintain a positive pressure in a face mask. However, heavy breathing demands momentarily an amount of air corresponding to up to 400 l/min or above. With the control according to the state of the art, an increase in airflow will lag behind and it will take too long time before the flow has increased, which increases the risk of leakage because of negative pressure in the face mask. In order to deliver an airflow of 400 l/min and above the size of the blower will be substantial, or it has to run at very high speed, resulting in heavy wear, and a high noise level.

Another disadvantage with existing PAPRs is the poor suction capacity which makes them unsuitable for use together with chemical filters, which have a much higher flow resistance compared to particle filters.

### THE INVENTION

One object of the invention is to provide a PAPR that mitigates or even eliminates the above drawbacks.

Another object of the invention is to provide a PAPR that is durable and have a higher overall efficiency.

Still another object of the invention is to provide a PAPR, in which the airflow can be regulated while using a blower running at constant speed, and a speed that is not so high that it causes substantial wear and a high noise level.

These and other objects of the invention are obtained with the breathing responsive powered air purifying respirator (PAPR) apparatus according to the invention, having the features stated in the characterizing part of claim 1.

Preferred and advantageous embodiments of the invention are defined in the dependent claims.

Thus, according to the invention a lobe blower is used for the supply of air to the face mask. Lobe blowers have excellent pressure head and suction head characteristics as well as the many other advantageous characteristics discussed in the specification.

In a preferred embodiment a timing belt drive (synchronous drive) is used to transfer the power from the electric motor powering the lobe rotor, which has several advantages. This drive creates very little noise. Compared with high precision gears, it is much less expensive, and still gives a very high precision drive.

Further, by using a by-pass regulator to adjust the airflow so that the required, predefined positive pressure is obtained in the face mask, an airflow corresponding to the max required airflow can be generated constantly by setting a corresponding motor rotational speed, and then adjusting the airflow to the face mask by recirculation of the purified air from the outlet to the inlet of the lobe blower. Another advantage is that the filters are not consumed unduly by producing an undue amount of purified air, despite the fact that the blower is running at constant speed.

According to one embodiment of the invention, the regulator is a pneumatically controlled regulator connected to the face mask through a hose, the active component being a diaphragm biased with a force corresponding to a predefined positive pressure in the face mask. When the pressure increases in the mask the regulator will open a by-pass valve, allowing recirculation of part of the flow of purified air from the outlet to the inlet of the blower.

### SHORT DESCRIPTION OF THE DRAWINGS

Further advantages and features of the invention will be evident from the following detailed description of embodiments of the invention with reference to the accompanying drawings, in which

FIG. 1 is a schematic view illustrating the working principle of the PAPR according to the invention,

FIG. 2 is a cross sectional view of the regulator in the PAPR shown in FIG. 1, and

FIG. 3 illustrates a timing belt drive of the lobe blower in the PAPR shown in FIG. 1.

### DETAILED DESCRIPTION OF THE INVENTION

The invention will now be described with reference to an embodiment of the invention illustrated in the accompanying drawings.

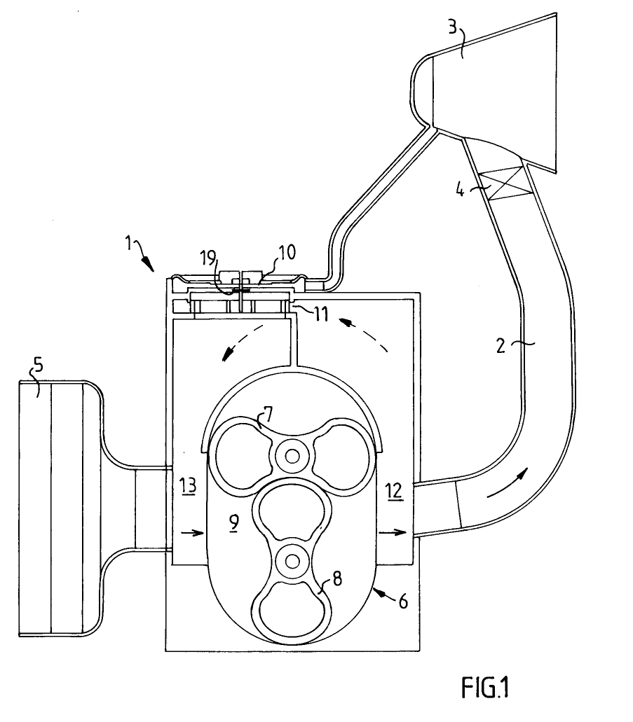

In FIG. 1 a PAPR, i.e. a powered air purifying respirator, is schematically illustrated and generally designated with the reference numeral 1. A hose 2 connects the PAPR to a face mask 3 equipped with a purge valve 4. Air is sucked into the PAPR via at least one filter 5. The PAPR may have more than one outlet, whereby it may also be connected to e.g. a chemical suit to establish a positive pressure therein to prevent leakage.

According to the invention, an airflow is generated with a lobe blower 6 having lobe rotors 7, 8, working in a blower housing 9. Each rotor may have two lobes, as illustrated, but could have three or four lobes as well. The lobe blower is powered by a battery powered electrical motor, which is not shown in the drawing for the sake of clarity. The lobe blower is operated at constant speed, while the airflow is regulated with the aid of a pressure regulator 10 mounted in a by-pass line 11 connecting the outlet 12 of the blower to its inlet 13. The direction of the airflow is indicated with arrows on the airflow lines.

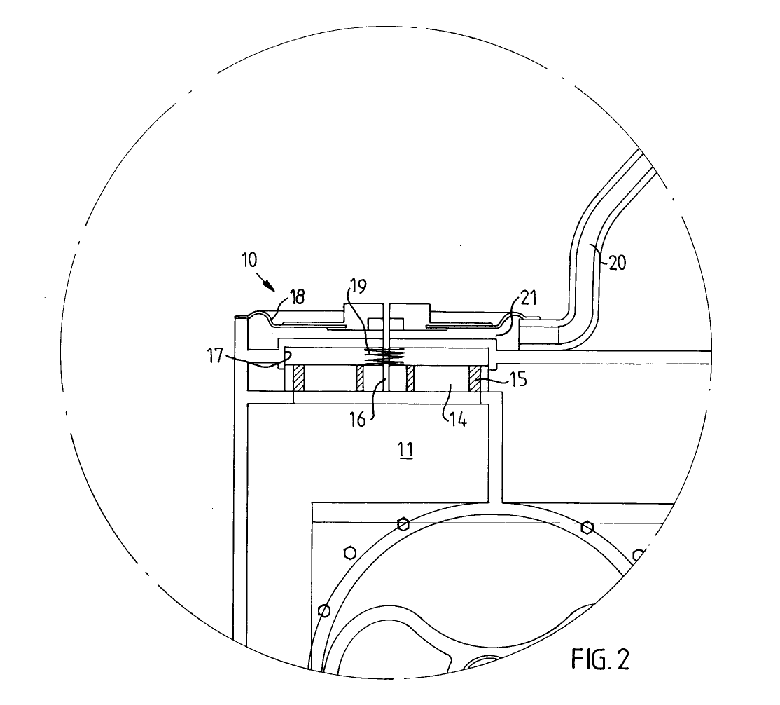

One object of the PAPR is to create a positive pressure in a face mask in order to prevent leakage into the mask. The regulator is connected to the face mask to regulate the pressure in the mask so that it is maintained substantially at a preset positive pressure. The regulator can be controlled in a number of ways, e.g. electrically, electronically, opto-electronically but also pneumatically, resulting in a simple, failsafe construction. In one preferred embodiment of the invention a pneumatically controlled regulator is used, and which will be described more in detail with reference to FIG. 2.

The object of the purge valve 4 is to make possible a ventilation of the PAPR before letting the airflow into the face mask. When the filter has been exposed to a hot environment, poisonous substances may have been released. The purge valve 4 may have three positions, one in which the airflow is vented into the atmosphere and a second in which the airflow is directed into the face mask, and a third in which the air flow line is completely shut off, when used together with a SCBA.

In FIG. 2 a cross sectional view of the regulator 10 is illustrated. A valve element 14 covers a valve opening 15 in the by-pass line. The valve element 14 has a rim 15 and a central part supporting a valve spindle 16, and has through openings there between. The valve element 14 slides in the lower opening of an orbital flange 17 of the regulator housing. The upper end of said orbital flange is covered with a diaphragm 18 which is biased against the opening of said orbital flange with a coil spring 19 or equivalent means. The biasing force of the spring coil equals the desired positive pressure in the face mask. The diaphragm is exposed on one side to ambient air pressure as the regulator works relative to ambient pressure.

When pneumatically controlled, a hose 20 with a diameter being less than the diameter of the airflow line for the purified air, connects the inside of the face mask to the interior 21 of the regulator underneath the diaphragm. When the positive pressure exceeds the biasing force of the spring coil 19, the diaphragm lifts and so does the valve element, letting more or less of the airflow free to enter the by-pass line 11.

If a small leakage is allowed between the rim of the valve element and the cylindrical flange, in which the valve element slides, there will be practically no friction between the moving parts, and no wear. By making the valve element with through openings, it is balanced and the only force that has to be overcome is the biasing force of the coil spring.

In another embodiment of the regulator valve element, not shown in the drawing, a ring of flexible material, e.g. silicone, having a cross section shaped as a “U” is used to seal around the valve opening into the by-pass line.

The lobe blower is inherently more silent compared to impeller blowers. Moreover it is more efficient and therefore the rotational speed therefore is be lower than for a impeller blower for the same airflow.

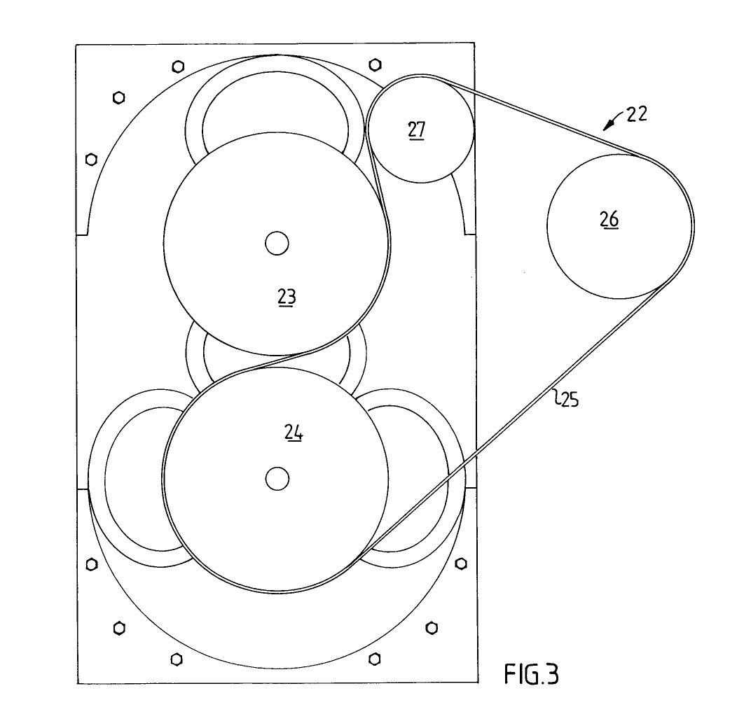

In FIG. 3 is schematically shown a timing belt drive 22, also called a synchronous drive. Each lobe rotor 7, 8 is connected to a respective pulley 23, 24. A timing belt 25, preferably having teeth on both sides (not illustrated in the drawing) is placed around the pulleys to rotate them in opposite directions. The timing belt is powered by an electrical motor, the drive pulley 26 of which is shown. Preferably, as is shown in the drawing the timing belt also runs over a pulley wheel 27, so that the timing belt will abut substantially the same amount of the pulleys 23, 24. Besides being silent, a timing belt drive is very exact, it has a low weight, and it is simple to exchange the belt and set the lobe rotors again. It would of course be possible to use a gear drive instead, but that requires high precision cut gear wheels, which will increase the costs, and also the noise.

Although not shown in the drawings, a check valve should be arranged in parallel with the regulator and the lobe blower, in case of power failure. The user then has the option of sucking air through the filter by breathing.

1. Breathing responsive powered air purifying respirator apparatus comprising an air-tight housing (11) having connections for filters (5) and for supply of purified air to a face mask (3), a motor-driven blower arranged inside said air-tight housing for drawing air through said filter units and for the supply of purified air into the face mask, characterized by: the blower being a lobe blower (6) delivering a substantially constant flow of air, a regulator (10) being arranged in said housing in parallel with the blower, a by-pass conduit (11) connecting the outlet side (12) of the lobe blower (6) to the inlet side (13) of the lobe blower (6), wherein said regulator is arranged to open said by-pass conduit when a pressure in the face mask exceeds a predetermined positive pressure.

2. Respirator apparatus according to claim 1, wherein the lobe blower (6) is powered by a battery powered electrical motor.

3. Respirator apparatus according to claim 1, wherein the power from the motor is transferred to the rotors through a timing belt drive (22).

4. Respirator apparatus according to claim 1, wherein the regulator (10) is pneumatically controlled.

5. Respirator apparatus according to claim 1, wherein the regulator (10) is comprised of a housing (11), a pressure sensitive diaphragm (18) arranged in the housing, one side being exposed to ambient pressure, and the other side being exposed the pressure prevailing in a face mask connected (20) to the respirator apparatus, the diaphragm being biased (19) to a closed position with a force corresponding to a predefined face mask pressure, and having a valve element (14), which opens against said biasing force, when the face mask pressure exceeds said predefined face mask pressure.

6. Respirator apparatus according to claim 1, also comprising a purge valve (4) arranged downstream the lobe blower, said purge valve having three positions, i.e. closed, venting the airflow from the lobe blower into the atmosphere, and connecting the airflow from the lobe blower to the face mask.
